(19) United States
(12) Patent Application Publication
(10) Pub. No.: US 2005/0284470 A1
(43) Pub. Date: Dec. 29, 2005

(54) Method and apparatus for micro-environment control

(76) Inventors: Chengping Wei, Gilbert, AZ (US); Linda Wei, Gilbert, AZ (US); Rena Li, Scottdale, AZ (US); Yong Shen, Scottdale, AZ (US)

Correspondence Address: CROWELL & MORING LLP;INTELLECTUAL PROPERTY GROUP, P.O. BOX 14300, WASHINGTON, DC, 20044-4300, US

(21) Appl. No.: 11/166,736

(22) Filed: Jun. 27, 2005

Related U.S. Application Data
(60) Provisional application No. 60/583,736, filed on Jun. 29, 2004.

(30) Foreign Application Priority Data
Jul. 7, 2004 (CN) 2004200840788

(51) Int. Cl. A61M 11/00; A62B 18/02

(52) U.S. Cl. 128/200.14

(57) ABSTRACT

A portable or wearable device and related methods of use that provide an altered microenvironment surrounding the respiratory path, specifically a person's mouth or nose, wherein the air in the microenvironment is either filtered to remove pollutants or pathogens, or wherein the air is altered by therapeutic or pharmaceutical or other agents, which disinfect the air or otherwise provide a desirable characteristic. In an alternative embodiment, a therapeutic or pharmaceutical agent is delivered to the air path to the individual wearing the device of the present invention.

### CROSS REFERENCE TO RELATED APPLICATIONS

This application claims the priority of U.S. Provisional Application No. 60/583,736, filed Jun. 29, 2004, the disclosure of which is expressly incorporated by reference herein.

### FIELD OF INVENTION

The invention is in the field of microenvironment control. More specifically, it relates to a miniature wearable electronic device that delivers filtered air, or air mixed with therapeutic or health-promoting agents directly to an individual's respiratory air-path.

### BACKGROUND OF THE INVENTION

Air pollution by chemical pollutants or microbial pathogens such as virus and bacteria is a major concern of modern life, especially in developing countries with lax environmental regulations or with high population density. Despite the advances in science and technology, filtration or the use of disinfectants, or a combination of both, remains the methods of choice for removal of pollutants and/or pathogens.

For example, stationary electrical devices are known in the art that vaporize perfumes or medicines into a room for de-odorant or sanitization or anti-microbial purposes. These devices usually contain a heater element, a container that holds evaporable chemical substance either in solid or liquid form (such as a solution in alcohol). When the heater element is connected to a household electricity outlet, heat is generated and helps evaporate the chemical substance into the air. When the carrier chemical evaporates, it carries the non-evaporable chemical particle with it. Those vaporized chemical particles flow out the device and circulate in the room with the air currents. Some devices have electrical fans to force the chemical vapor out of the device into the room.

The chemicals used are either deodorants or disinfectants to kill bacterial, viral and other pathogens in the air. In order to be effective, the concentration of the chemicals in the air must reach certain level, thus these devices must contain enough volume of the chemicals and have high enough air flow rate in order to reach such an effective concentration. Accordingly, the stationary evaporating devices can be only used in relatively small enclosed environments, and are largely ineffective in open environment such as outdoors or on the street in a city. Their effectiveness is also limited in large enclosed environments such as airports and train stations.

Wearable, passive filters such as masks and covers made of filter paper or cloths are somehow effective, yet their use by the population at large is disfavored because of lack of comfort and aesthetics.

Portable active air purification devices are also known in the art. For example, U.S. Pat. No. 5,484,472, to Weiberg, discloses a device used to detoxify and circulate air around a person's face. The device electronically provides a corona discharge (ozone) to destroy pollutants. These devices are described as miniature electronic air purifier, clipped to a wearer's front shirt pocket or hanged from her neck. This device produces a corona discharge around an emitter point connected to a DC power supply. A small DC fan draws air which is exposed to the corona discharge. The corona discharge generates ozone and negative ions that is delivered to the individual's face. A filter may also be used in the device to remove the ozone. Trace amounts of ozone, however, remains and can irritate the respiratory system. This device does not supply pharmaceutical or therapeutic or other chemicals stored in a medium in the device. In addition, these devices create electricity discharges that may spark fires in areas containing flammables such as a gasoline station.

Therefore there is a need for wearable environment controlling or conditioning devices that avoid the drawbacks in the prior art.

### SUMMARY OF THE INVENTION

The present invention provides a portable or wearable device and related methods of use that provide an altered microenvironment surrounding the respiratory path, specifically a person's mouth or nose, wherein the air in the microenvironment is either filtered to remove pollutants or pathogens, or wherein the air is altered by therapeutic or pharmaceutical or other agents, which disinfect the air or otherwise provide a desirable characteristic. In an alternative embodiment, a therapeutic or pharmaceutical agent is delivered to the air path to the individual wearing the device of the present invention.

The present invention provides a portable or wearable device and related methods of use that provide an altered microenvironment surrounding the respiratory path, specifically a person's mouth or nose, wherein the air in the microenvironment is either filtered to remove pollutants or pathogens, or wherein the air is altered by mixing with one or more therapeutic or pharmaceutical or other agents, which disinfect the air or otherwise provide a desirable characteristic. In an alternative embodiment, a therapeutic or pharmaceutical agent is delivered to the air path to the individual wearing the device of the present invention.

In one embodiment, a portable device of the invention comprises a micro-air-processor, and an attachment mechanism that permits the device to be worn by the person, wherein air in the microenvironment is either filtered to remove pollutants or pathogens, or wherein air is altered by filtration or by mixing with one or more chemical substance or both. In another embodiment, the device further comprises a control mechanism.

The micro-air-processor may comprise a filter means coupled to an electric fan wherein the fan drives air through the filter to purify the air. The micro-air-processor may further comprise a storage medium that stores one or more chemical substance, and wherein the chemical substance is released when the device is in operation. The storage medium may be coupled to an electric heating device which facilitates release of the chemical agent.

In another embodiment, the control mechanism of the device of the present invention further comprises at least one sensor that can determine the person's body temperature, existence of a chemical, or air flow rate from the micro-air-processor. Preferably, the control mechanism may comprise a user control interface, wherein air flow rate, or a ratio of the chemical substance with the air in the microenvironment is controlled. The user control interface comprises a display, an input means, and optionally a serial port. The ratio may be controlled via controlling the heating element or the fan.

Preferably, the micro-air-processor, the at least one sensor, the storage medium and the fan are encased in a housing which has at least one air intake opening, and at least one air output opening.

Suitable chemical substance for the present invention may be one or more pharmaceutical agents, air-refreshing agents, herb-based substances, or a mixture thereof.

According to another embodiment of the invention, the attachment mechanism comprises a clip or a means for attaching the micro-air processor to the ear, nose, or face of a person. Specifically, the attachment mechanism may comprise a clip or a means for attaching the micro-air processor to the ear, nose, or face of a person., and a means for separately attaching the control mechanism to the person.

In a simplified embodiment, the device of the invention has micro-air-processor that does not contain a filtering or electric fan function. A storage medium is provided that stores one or more chemical substance, and the chemical substance is released when the device is in operation. Preferably, the chemical substance is in liquid or gas form stored in a pressurized container and released by spraying, for example via a user controlled micro-actuator.

The present invention further provides a method for altering a microenvironment in the vicinity of a person's respiratory air path, the method comprising: (1) altering via filtering air, or mixing into the air a chemical substance, and (2) actively or passively delivering the altered air to the vicinity of respiratory path. Preferably, the air is delivered to the vicinity of the person's mouth or nose, e.g. via a portable device of the present invention.

### BRIEF DESCRIPTION OF THE DRAWINGS

FIG. 1 is a block diagram of a micro environment controller according to one embodiment of the invention.

FIG. 2 is a perspective view of a complete micro environment controller according to one embodiment of the invention.

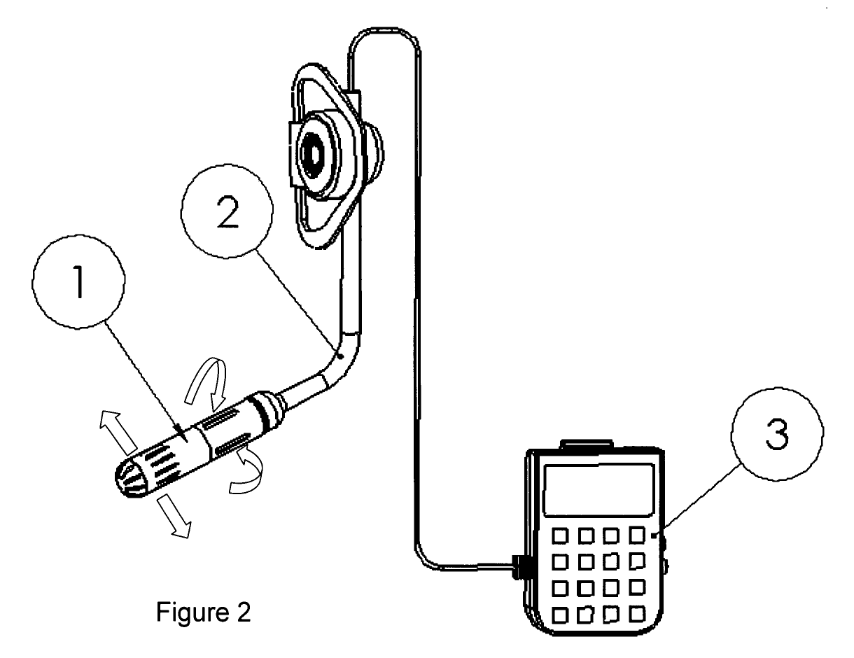

FIG. 3 is an exploded view of an air processor according to one embodiment of the invention, illustrating the process of air purification or alteration.

FIG. 4a is a perspective view of an air processor, and FIG. 4b shows a cut away view of the micro air processor according to one embodiment of the invention.

FIG. 5 is another exploded view of the micro air processor according to one embodiment of the invention.

FIG. 6 is a block diagram of a controller according to one embodiment of the invention.

FIG. 7a and 7b are two perspective views, and 7c shows an exploded view of a controller according to one embodiment of the invention. 7b shows a controller with a clipper.

FIG. 8a shows the top side, and FIG. 8b shows the bottom side of, the printed circuit board of a controller according to one embodiment of the invention.

FIG. 9a shows an attachment mechanism for an embodiment of an air processor. FIG. 9b is a exploded view showing details of the attachment mechanism

FIG. 10 shows an individual wears a micro environment controller.

FIGS. 11a and 11b show another embodiment of an environment controlling device of the present invention.

FIG. 12 is an exploded view of the device shown in FIG. 11.

FIG. 13 illustrates how the device in FIG. 11 is attached.

### DETAILED DESCRIPTION OF PREFERRED EMBODIMENTS

The present invention provides methods and devices that can be used for air purification, air refreshing, air pollutant removal, delivery of pharmaceutical or therapeutic agents. The present invention provides a miniature microenvironment controlling device that comprises a wearable micro-air-processor, an air flow and agent mixing ratio controller connected to the micro-air processor, and an attachment mechanism that permits the device to be worn by an individual. The controlling device controls the microenvironment surrounding the individual's respiratory path by filtering the air and/or mixing the air with a therapeutic or pharmaceutical agent in the microenvironment.

Preferably, the device operates on ordinary alkaline or rechargeable batteries.

The miniature microenvironment controlling device of the present invention preferably includes one or more temperature, chemical, and air flow velocity sensors to monitor the individual's body temperature, or air/chemical mixing ratio, especially the ratio in the vicinity of the person's respiratory path, and air flow volume.

In one embodiment, the miniature microenvironment controlling device of the present invention comprises a controller that controls for example air flow or agent mixing ratio. The controller comprises a user control interface, a display and input keys for reading the monitored data, settings, and altering the controlling condition. Furthermore, the controlling device may also include a digital serial interface (e.g. a RS232 or USB or IrDA) (spell out) that allows the user to link the device to a personal computer to download monitored conditions, which may be used for professional analysis.

Many wearing mechanisms are known to those skilled in the art. For example, any of those mechanisms used for personal electronic devices, e.g. a clip, a band made of Velcro can be used for the control mechanism of the device of the present invention. The control mechanism may also be simply carried around by the person in their hands or pockets. The micro air processor may be worn as a wearable microphone or mouth piece commonly used by performers or speakers, via attachment to the person's ears, nose, or as a pair of eye glass with suitable extension that reaches the mouth and/or nose of the person.

Referring to the figures, preferred embodiments of the device of the present invention are described in more details below.

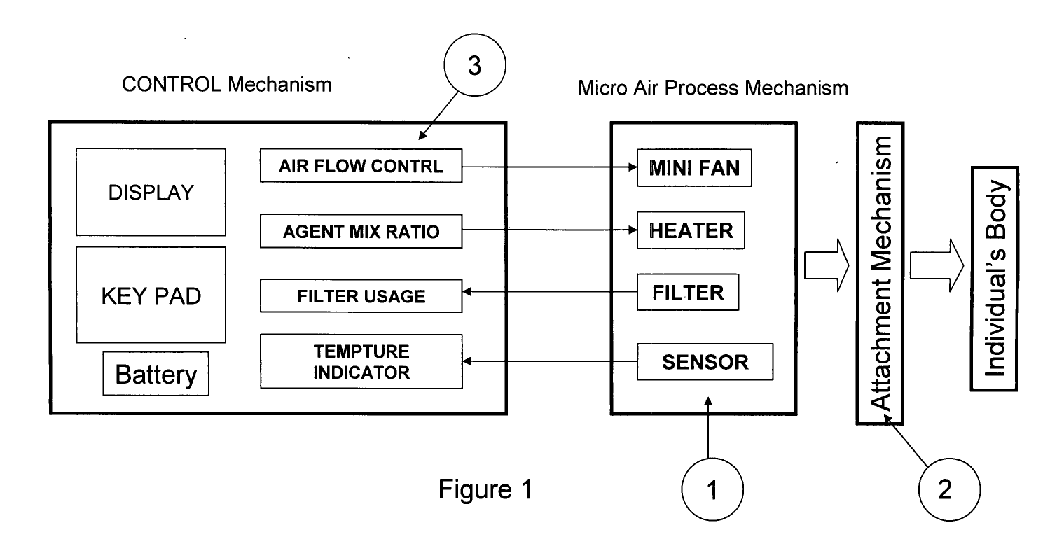

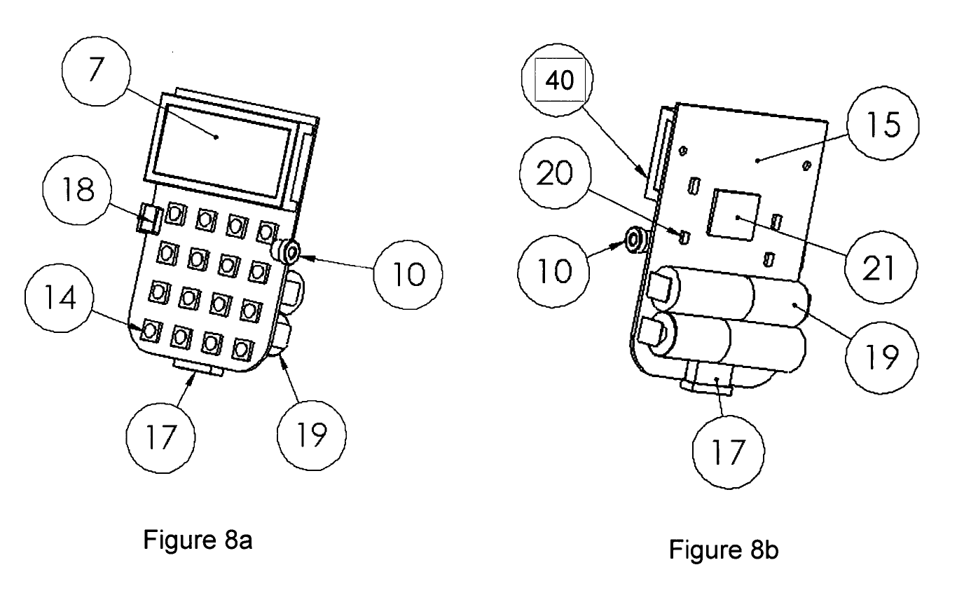

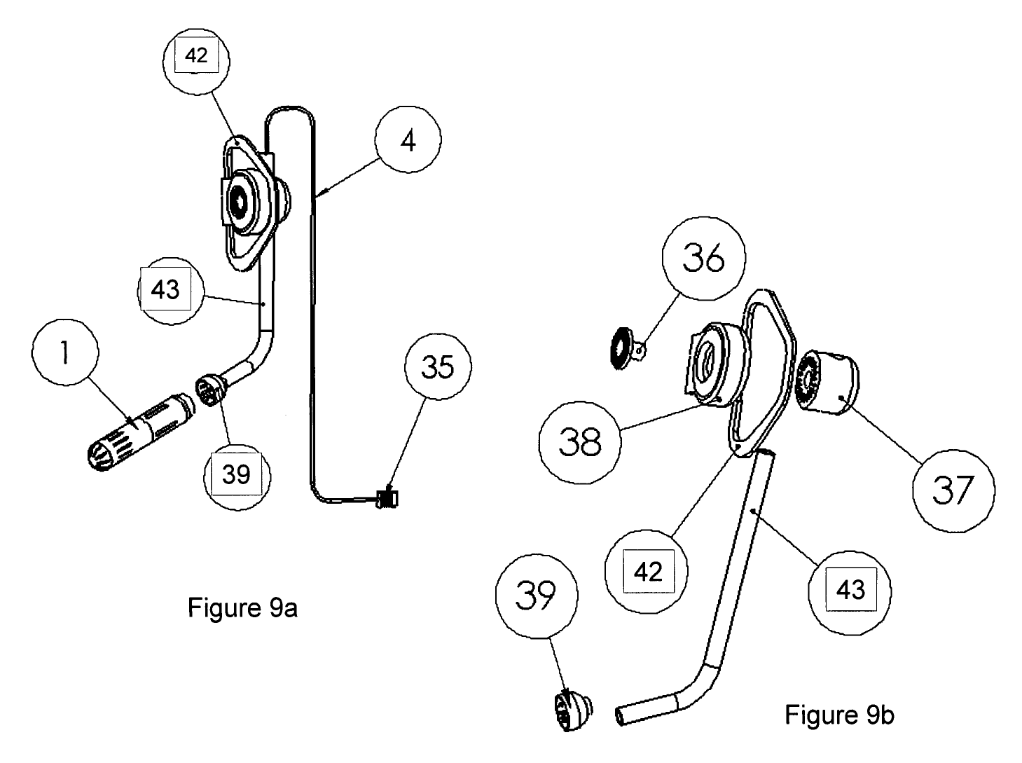

As depicted in FIG. 1, the wearable microenvironment control apparatus of the present invention comprises a Micro-Air-Process Mechanism 1, a Control Mechanism 3, and an Attachment Mechanism 2. The Micro-Air-Process Mechanism 1 (which will be described in more detail below) is attachable to the person or clothing, via the Attachment Mechanism 2, of the individual such that it is in close proximity of the respiratory air path of the individual. The Attachment Mechanism 2 may rest on the nose or ear of the individual who wears the apparatus of the invention. As discussed above, many such attachment mechanisms are well-known to those skilled in the art, and are commonly used for earphones, microphones, eyeglasses etc. The Micro-Air-Process Mechanism 1 is connected via a flexible electrical cable 4 (see e.g. FIG. 9) to the Control Mechanism 3 which comprises a power supply, for example in the form of one or more batteries 19 (FIG. 8).

The Micro-Air-Process Mechanism (MAP) 1 for the apparatus of the present invention may perform (1) a purifying function for removing pollutants from the air via a suitable filter, and delivering the purified air to the proximity of the nose or mouth of the individual who wears the apparatus, (2) a delivery function delivering air (which may be optionally filtered first) mixed with one or more therapeutic or disinfecting agents to the proximity of the nose or mouth of the individual, or (3) a condition-monitoring function monitoring the individual's body temperature, air flow and agent/air mixing ratio. Preferably, two or more of the above functions are performed.

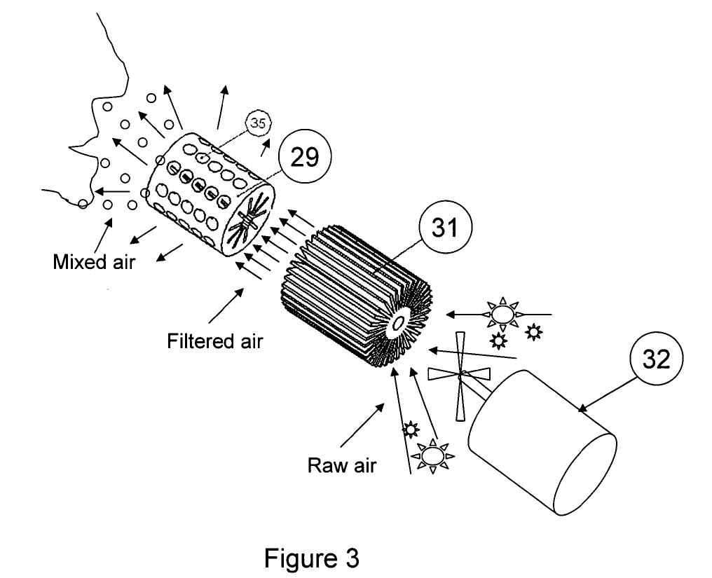

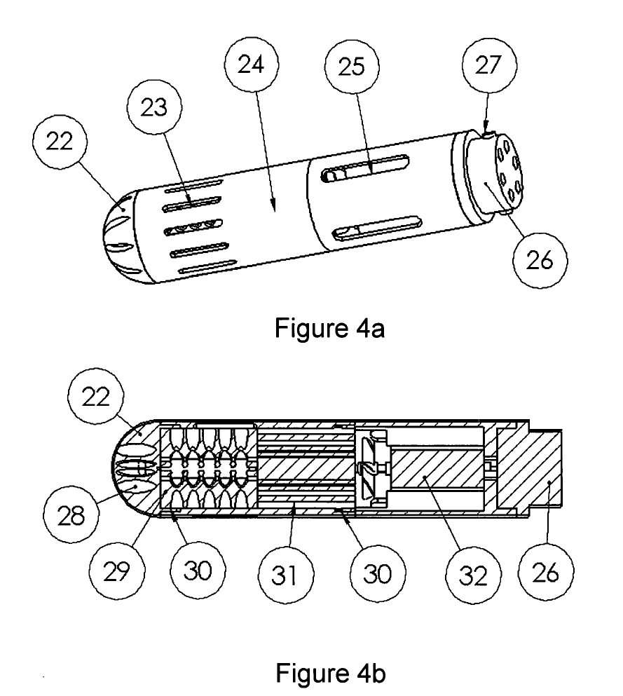

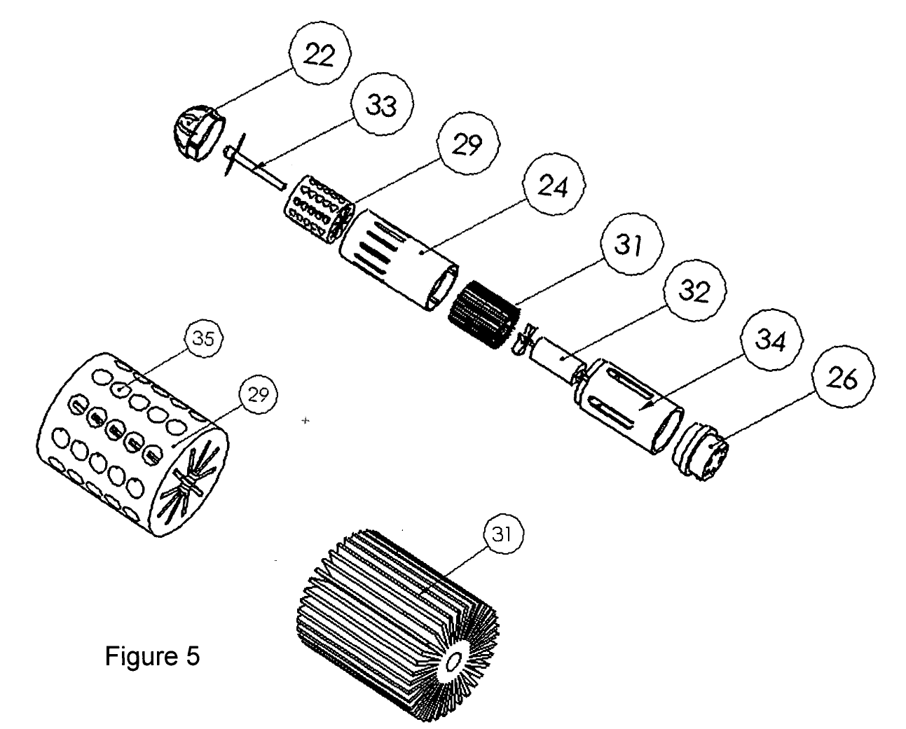

In one embodiment, the MAP comprises a medium 29 suitable for storing a chemical agent, a filter 31, a fan 32 (FIG. 3), and molded plastic housing 22, 24 (FIG. 4), 34 (FIG. 5). One skilled in the art will recognize that many suitable filters, storage media and miniature electric fans (preferably operable with DC power) are available and can be used for the present invention.

As shown in FIG. 4 and FIG. 5, housing 22, 24, 34, which may be made of molded plastic (ABS) or a suitable metal material, may comprise three sections: the mini-fan section 32, 34, the filter and chemical agent section 24, 29, 31, and the sensor/heater section 22, 33. Each of the sections is connected from one to another, for example with latches 12 on the edge of the housing. Metal tabs 30, on the ends of each section's housing and connected by the embedded electrical wires (not marked) embedded in the housing, provide electrical connections from the sensor/heater section 22, 33 through the filter/agent section 24, 29 31, to the connector 26 located at the end of the mini fan section 32, 34. The sensor/heater assembly 33 comprises a sensor and optionally a hecter for facilitating evaporation of the chemical agent, may be a piece of resistive wire, wound in a cylindrical shape and soldered on the metal tabs on the molded plastic cup which may be a sensor for temperature, flow viscosity, or a particular chemical compound, either in the environment or to be released, can also be soldered on the rest of metal tabs on the molded plastic cup. Many such sensors are known and readily available in the art. Windows 28 may also be provided on the plastic cup 22. Such windows are particularly suitable for infrared temperature sensors, for use for example in detecting the body temperature of an individual who wears the device.

In one embodiment, the storage medium 29 may contain a large, centrally located hole into which the heater 33 is inserted. Chemical agent may be added or replenished to the storage medium after the medium is in place, or the entire storage medium may be replaced as a refill. A plurality of smaller holes 35 may be provided in the storage medium around the center hole to provide air paths.

Filter 31, which is preferably replaceable, is held in the housing 24 behind the chemical agent 35. In the embodiment shown in FIG. 5, it is folded in a star shape to provide large surface area for removing pollutants and other particles from incoming air. It may be a conventional fiber-based filter, or electro-statically charged.

An electrostatic air filter is known to those ordinarily skilled in the art. For example, it may use electro-statically charged polypropylene and polyurethane filtration materials, and can trap particles as small as 0.3 μm. The static charge attracts and traps airborne particles of the opposite charge. The electrostatic air filter can be replaced with a new one or reused after washing or otherwise removing the trapped particles. Other type of filter, such as cotton gauze, foam, and paper filter can also be used in the MAP.

It is readily recognized that storage medium 29 and filter 31 should have a slightly smaller diameter than the inner diameter of the housing 24, so as to be able to slide in and out for refilling and replacement. A plurality of openings or windows 23 are provided on the housing 24 to allow processed air out to one's respiratory air path.

A mini fan is located further behind on the MAP 1. Fan motor 32 is preferably a miniature DC motor, having a diameter smaller than the inner diameter of the housing 34. The mini fan is held in place by plastic supporting structures on the inner surface of the housing. Adjacent to the motor holding structures there are a plurality of air intake openings 25, allowing air flowing in to the MAP 1 for processing.

FIG. 9 shows one embodiment of the attachment mechanism for the apparatus of the present invention. It provides a mechanism to hold the MAP 1 near the individual's respiratory path. It may also function to provide an electrical connection between the MAP 1 and control mechanism 3.

One embodiment of the attachment mechanism (AM) 2 is depicted in FIG. 9, which is in the form of an ear piece. A ring-shaped or semi-circle structure 42 hangs the apparatus to an ear, and via an extender 43, holds the MAP in the proximity of the individual's respiratory air path. An electrical connector 39 on one end of the extender 43 connects to the MAP 1, and flexible cable 4, embedded in the plastic extender 42, extends from the other end of the extender and terminates in an electrical plug 35, for plugging into a corresponding connector on the controller 3. In FIG. 9a, the AM 2 is shown with the MAP 1, where structure 42 is approximately semicircle in shape. Two cylindrically shaped parts 37 and 38, can be screwed together, optionally with a thumbnail screw 36, holding the semicircle structure 42 in place. Part 37 has a cut out which his slightly smaller than the diameter of the adjustable extender to hold the extender in place. Screw 36 may be adjustable and when loosened may allow the extender 43 to be telescoped out or retracted in for different length requirement and the extender can also be rotated for different angle and bent for different height requirements. When tightened, the screw fixes the extender's position.

Electrical connector 39 may be a conventional twist on connector well known in the art. It provides the electrical connection and mechanical support for the MAP 1.

The control mechanism (CM) for the apparatus of the present invention may provide one or more of the following functions: (1) controlling the air flow of the MAP 1; (2) controlling the chemical to air mixing ratio; and (3) provides a user interface, wherein a display 40 shows e.g. the age of the filter 31, current body temperature, chemical mixing ratio and battery level etc., a key pad 6 allows the individual to set operation conditions such as air flow, chemical mixing ratio, or engage in a data downloading operation.

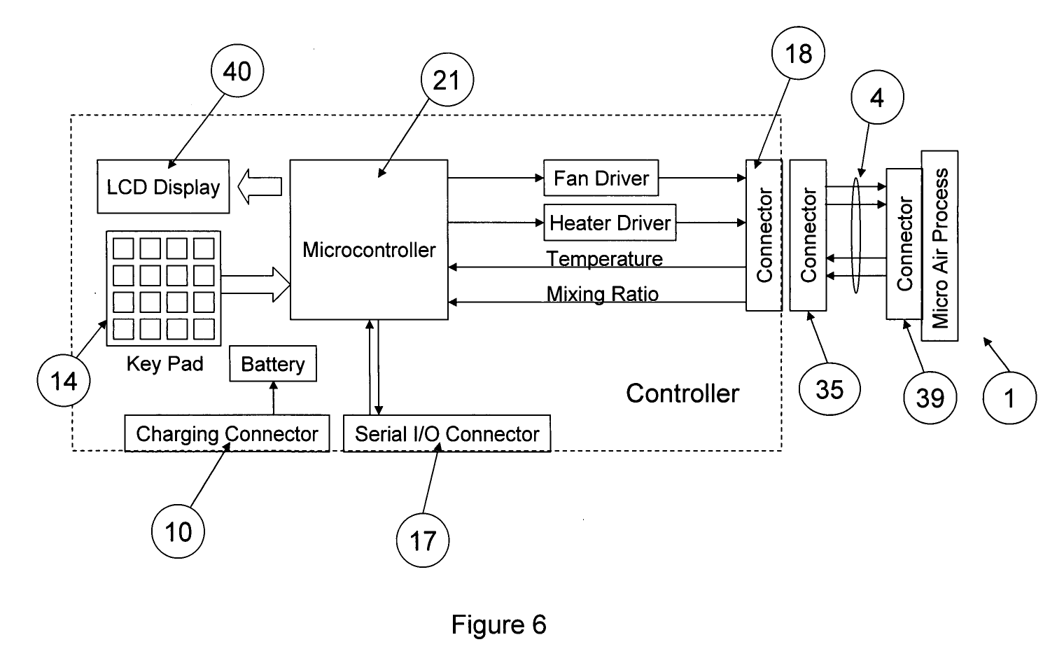

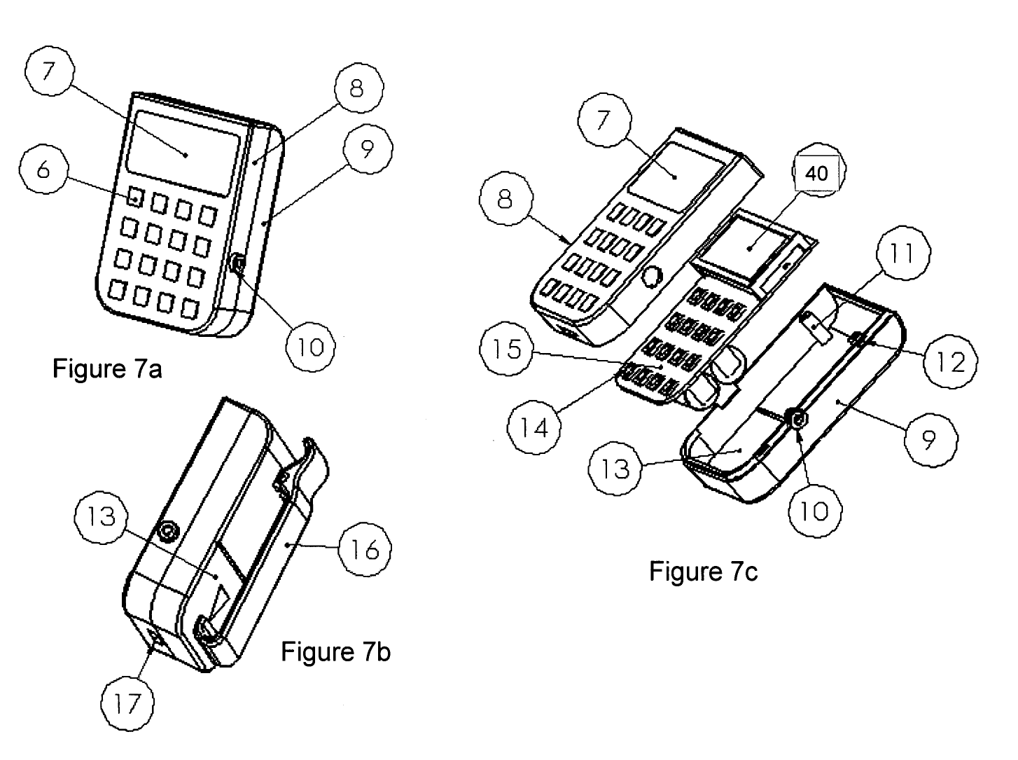

FIGS. 6 and 7 depict one embodiment of the CM, which comprises housing 8, 9, a printed circuit board (PCB) 15, and a battery or batteries 19 (alkaline or rechargeable). A LCD display assembly 40 is mounted on the board 15. Each of the connector 18 for connecting to the MAP, the charger connector 10 and serial data connector 17 is located on one side of the PCB 15 (e.g. left, right and bottom side, respectively). A switch assembly is located on the top of the PCB below the LCD assembly. On the top half of the hosing, a window 7 is provided which may be covered by transparent plastic for showing the display. A molded plastic key pad 6 is affixed in place on the top half housing 8 below the display window. A microcontroller integrated circuit 21, transistors, and other passive and active components are provided, e.g. soldered, on both top and bottom of the board 15. On the bottom half of the housing 9 there is a removable door 13 for battery 19 replacement. The printed circuit board 15 is secured, e.g. by two molded plastic posts, and sandwiched in between the top and bottom housing 8, 9 assemblies. The top and bottom housing assemblies 8, 9 are held together, e.g. by the molded latches 12 on the edges of the top and bottom plastic housing assemblies 8, 9.

When batteries 19 are connected, the microcontroller is powered, and runs in the standby mode. Once the power key (on the key pad) is depressed, the microcontroller turns on the mini fan, and puts a current through the heater 33 to activate the chemical agent 29. The fan speed (air flow) and heating current level are determined by default settings that may be programmed in the read only memory ROM inside of the microcontroller 21. These setting can be altered by the individual. When the micro environment controller is turned on, air is drawn into the MAP 1 through the inlet windows 25. The air then is forced through the filter 31. Pollutants and dusts are trapped by the filter 31. At the same time chemicals in the storage medium is heated up by the heater 33 and starts to vaporize. The chemical vapor then is pushed through air paths out of the MAP's outlet windows 23, along with filtered air into the individual's respiratory path, in the proximity of nose and mouth. Optionally, the mixture (chemical/air) can be monitored and feedback signals are sent to the microcontroller to maintain a constant mixing ratio.

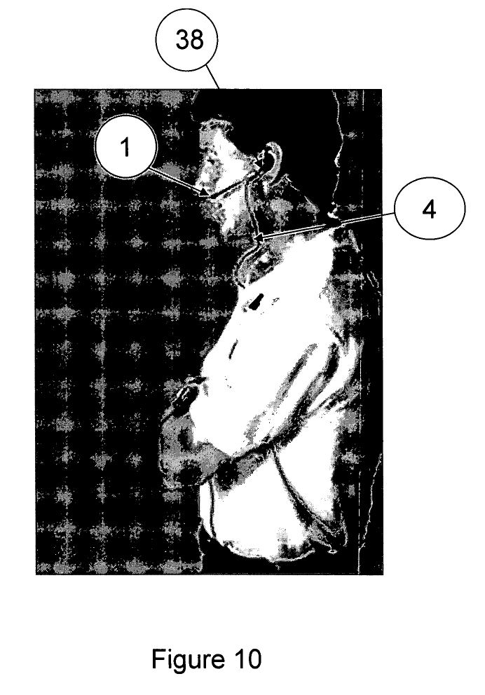

With a clip 16, or other similar mechanisms, the CM 3 can be easily clipped on the belt, or be placed inside an pocket. An individual can wear the micro environment controller in the same fashion as wearing an earphone (FIG. 10).

Depending on each individual's needs, one can use the environment controllers to neutralize a foul odor with a perfume, others may use micro environment controllers for delivering a pharmaceutical agent for medical conditions (i.e. for the treatment of asthma) without affecting others in the vicinity. Optionally, the micro environment controller can be used to deliver pharmaceutical agents in three different modes; (1) constant mode, wherein the MAP continuously delivers the pharmaceutical agent as long as the device is turned on, (2) timed mode, wherein the device activates and delivers the pharmaceutical agent in a timely manner, and (3) on-demand mode, wherein the amount of delivered pharmaceutical agent and the delivery length and time can be preprogrammed by the individual, and the pharmaceutical agent activation and delivery are trigged manually or automatically by the individual's health condition such as an asthma attack. The amount of delivered pharmaceutical agent and the delivery length may further be determined by the individual's health condition (i.e. body temperature, breathing pattern etc.)

In public locations, such as in the airport, at the train station where the environment can not be easily controlled, the micro environment controller can be very effective whether it is used for deodorant or for airborne transmitting disease control. For example, during the flue season, people can wear the micro environment controller filled with anti-viral agents to prevent from getting or transmitting the flu virus.

Alternatively, the micro environment controller can be used to monitor the individual's body temperature continuously. The IR temperature sensor in the MAP detect the individual's body temperature and digitize it and store the digital temperature data with a time stamp in the RAM (Random Access Memory) in the microcontroller. The display on the controller (CM) displays the current body temperature and the individual can download the stored digital body temperature data to a personal computer for analysis through the serial data connector.

At any time, the individual can detach the MAP 1, clean it, and reload a new chemical agent for different purpose. The controller can display the filter's age and remind the individual when the filter 29, 31 should be replaced.

After a days' use, the individual can place the micro environment controller into a charger to recharge the batteries 19 for next day's use.

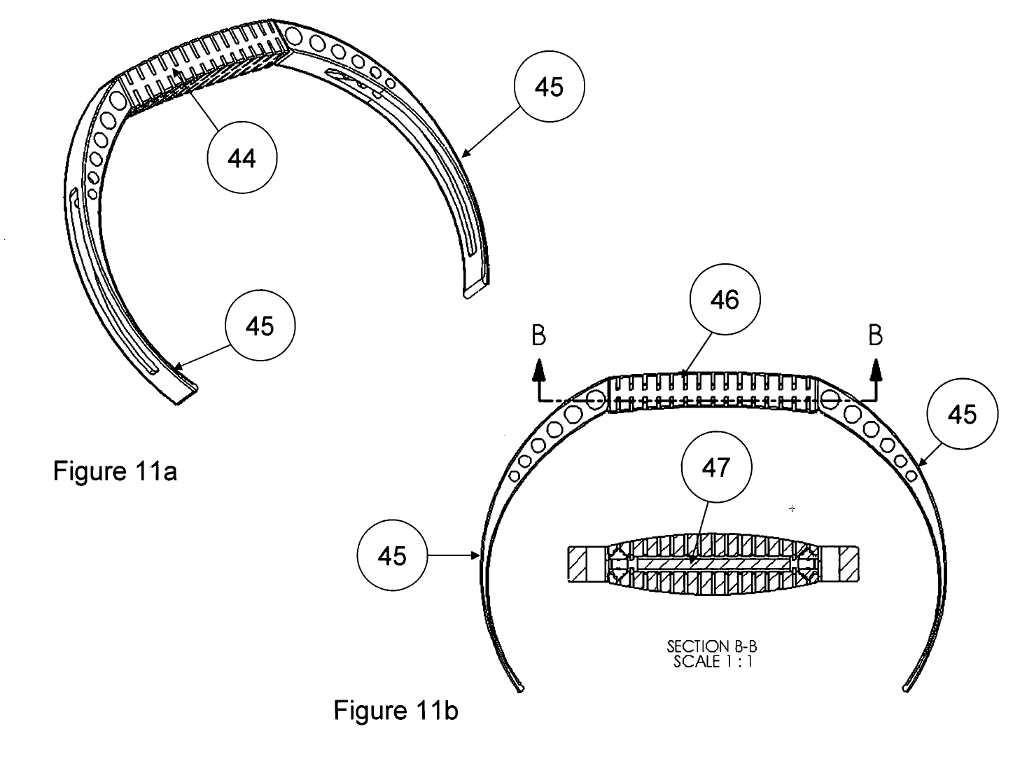

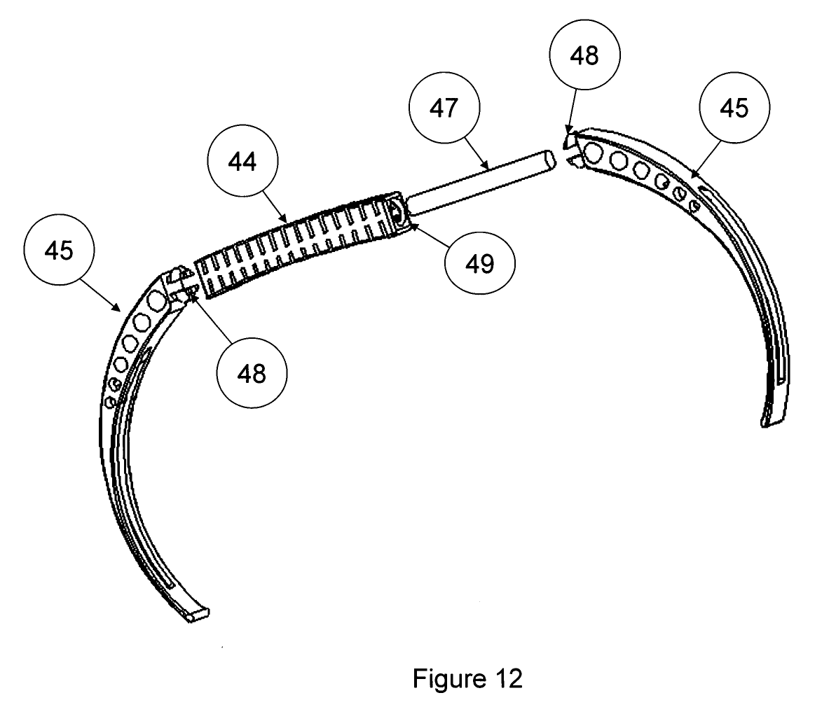

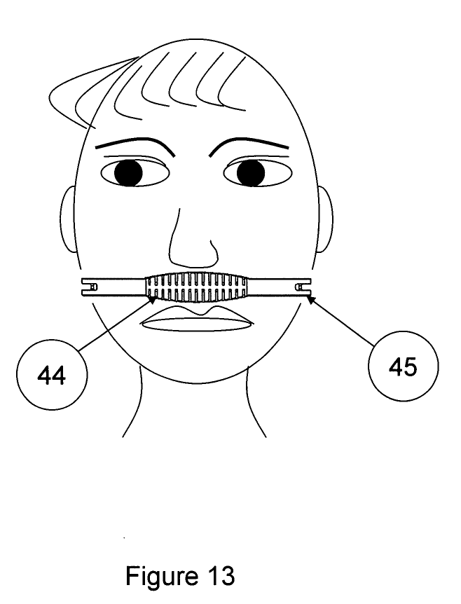

In another embodiment, the micro environment control device is simplified, as shown in FIG. 11. The device comprises three parts (see FIG. 12); a housing (such as a molded plastic container) 44, with at least a plurality of venting holes 46 containing a vaporizable chemical agent 47 but no filter or any electrical function; and two legs 45, latched 48 on openings 49 on each end of housing 44, for attaching the container to the individual's face in or around the respiratory path, as shown in FIG. 13.

The individual's natural breathing air flow carries the chemical vapor out of the agent's container 44 and the vapor alters the micro environment in the individual's respiratory path. Different therapeutic or pharmaceutical or other types of agents can be inserted in the device for different purposes. One or both legs 45 of the device can be removed for placing or replacing the chemical agent 47.

Therapeutic or pharmaceutical agents suitable for the present invention may be any type, such as perfume, herbs or herbal extracts or preparations, anti-viral compounds, antibacterial compounds, other drugs, antibiotics. The agent, mixed with evaporable chemicals, can be in a solid or liquid form. The releasing methods can be via vaporizing or spraying.

When a spraying method is used, the heater assembly 33 will be replaced by an electro-magnetic actuator. A chemical agent in liquid or gaseous form is stored in a pressurized mini-container. When the nozzle at the top of the mini bottle is depressed by the electro-magnetic actuator, s chemical agent mist is released out of the bottle and the air flow created by the fan then delivers the processed air to the individual's face in the respiratory path.

The foregoing description and examples have been set forth merely to illustrate the invention and are not intended to be limiting. Since modifications of the disclosed embodiments incorporating the spirit and substance of the invention may occur to persons skilled in the art, the invention should be construed broadly to include all variations and equivalents falling within the scope of the appended claims and equivalents thereof. All references cited hereinabove and/or listed below are hereby expressly incorporated by reference.

1. A portable device for altering a microenvironment near a respiratory path of a person, the device comprising a micro-air-processor, and an attachment mechanism that permits the device to be worn by the person, wherein air in the microenvironment is either filtered to remove pollutants or pathogens, or wherein air is altered by filtration or by mixing with one or more chemical substance or both.

2. A device according to claim 1, wherein the device further comprises a control mechanism.

3. A device according to claim 2, wherein the micro-air-processor comprises a filter means coupled to an electric fan wherein the fan drives air through the filter to purify the air.

4. A device according to claim 3, wherein the micro-air-processor further comprises a storage medium that stores one or more chemical substance, and wherein the chemical substance is released when the device is in operation.

5. A device according to claim 4, wherein the storage medium is coupled to an electric heating device which facilitates release of the chemical agent.

6. A device according to claim 5, wherein the device further comprises at least one sensor that can determine the person's body temperature, existence of a chemical, or air flow rate from the micro-air-processor.

7. A device according to claim 6, wherein the control mechanism comprises the sensor, and further comprises a user control interface, wherein air flow rate, or a ratio of the chemical substance with the air in the microenvironment is controlled.

8. The device according to claim 7, wherein the ratio is controlled via controlling the heating element or the fan.

9. The device according to claim 3, wherein the micro-air-processor, the at least one sensor, the storage medium and the fan are encased in a housing which has at least one air intake opening.

10. A device according to claim 9, wherein the housing comprises at least one air output opening.

11. A device according to claim 1, wherein the chemical substance is one or more of a pharmaceutical agent, an air-refreshing agent, or a herb-based substance.

12. The device according to claim 7, wherein the user control interface comprises a display, an input means, and optionally a serial port.

13. The device according to claim 1, wherein the attachment mechanism comprises a clip or a means for attaching the micro-air processor to the ear, nose, or face of a person.

14. The device according to claim 2, wherein the attachment mechanism comprises a clip or a means for attaching the micro-air processor to the ear, nose, or face of a person., and a means for separately attaching the control mechanism to the person.

15. A device according to claim 2, wherein the micro-air-processor further comprises a storage medium that stores one or more chemical substance, and wherein the chemical substance is released when the device is in operation.

16. The device according to claim 15, wherein the chemical substance is in liquid or gas form stored in a pressurized container and released by spraying.

17. A method for altering a microenvironment in the vicinity of a person's respiratory air path, the method comprising: (1) altering via filtering air, or mixing into the air a chemical substance, and (2) actively or passively delivering the altered air to the vicinity of respiratory path.

18. The method according to claim 17, wherein the air is delivered to the vicinity of the person's mouth or nose.

19. The method according to claim 17, wherein the air is altered via a portable device.

20. The method according to claim 19, wherein the portable device comprises a micro-air-processor, and an attachment mechanism that permits the device to be worn by the person, wherein the air in the microenvironment is either filtered to remove pollutants or pathogens, or wherein the air is altered by filtration or by one or more chemical substance or both.

21. The method according to claim 20, wherein the device further comprises a control mechanism.

22. The method according to claim 21, wherein the micro-air-processor comprises a filter means coupled to an electric fan wherein the fan drives air through the filter to purify the air.

23. The method according to claim 17, wherein a pharmaceutical composition is delivered to a person in need thereof.
